(19) United States
(12) Patent Application Publication
(10) Pub. No.: US 2013/0190844 A1
(43) Pub. Date: Jul. 25, 2013

(54) PHOTO-STIMULATION METHOD AND DEVICE WITH LIGHT MIXTURE

(71) Applicants: Ming-Chieh TU, Keelung City (TW); Yi-Wei Hsiao, New Taipei City (TW); Chung-Pei Lee, Taipei City (TW); Jung-Chien Chang, New Taipei City (TW); Yu-Chia Tsao, Taipei City (TW)

(72) Inventors: Ming-Chieh TU, Keelung City (TW); Yi-Wei Hsiao, New Taipei City (TW); Chung-Pei Lee, Taipei City (TW); Jung-Chien Chang, New Taipei City (TW); Yu-Chia Tsao, Taipei City (TW)

(73) Assignee: Forward Electronics Co., Ltd., Taipei City (TW)

(21) Appl. No.: 13/470,619

(22) Filed: May 14, 2012

(30) Foreign Application Priority Data
Jan. 19, 2012 (TW) 101102138

(51) Int. Cl. A61N 5/06

(52) U.S. Cl. 607/90; 607/88

(57) ABSTRACT

Disclosed is a photo-stimulation method and device with a light mixture. The method includes the following steps: providing a light-emitting diode (LED) illuminant which is a combination of a yellow LED and a red LED; and illuminating a subject by the LED illuminant to promote collagen synthesis, to suppress microbial growth, or to inhibit melanin synthesis, wherein the yellow LED is in an illuminance range from 1,000 to 3,500 lux, the red LED is in an illuminance range from 6,000 to 9,500 lux, and the number ratio of the yellow LED to the red LED is 0.5-2:0.5-2.

### CROSS REFERENCE TO RELATED APPLICATION

This application claims the benefits of the Taiwan Patent Application Serial Number 101102138, filed on Jan. 19, 2012, the subject matter of which is incorporated herein by reference.

### BACKGROUND OF THE INVENTION

1. Field of the Invention

The present invention relates to a photo-stimulation method and a photo-stimulation device with a light mixture and, more particularly, to a photo-stimulation method and device with a light mixture, which can promote collagen synthesis.

2. Description of Related Art

After dermatological diagnosis, drugs are generally used to treat patients' skin conditions, such as acne. However, drug therapy frequently incurs side effects and long-term drug administration also results in metabolic loads on patients. Notably, such therapy does not bring desirable efficacy of treatment and treated patients often have a relapse of skin conditions. Hence, patients' skin conditions can not be efficiently eradicated.

In recent years, medical cosmetology has been greatly developed. Some research reported that blue light with a wavelength of 400-475 nm could be applied to treat acne. After blue light illumination, inflammation and red and swollen conditions of tissues caused by acne can be alleviated because photosensitive coproporphyrin in Propionibacterium acnes or tissue cells reacts with blue light to form toxic singlet oxygen and free radicals which kill bacteria and some cells of the sebaceous gland. In addition, red light with a wavelength of 600-750 nm, yellow light with a wavelength of 550-600 nm, and green light with a wavelength of 500-570 nm can stimulate fibroblasts in the dermis to induce synthesis of collagen and to prevent skin aging.

At present, in order to achieve the above-mentioned effects, laser or intense pulsed light is often applied in the industry of medical cosmetology. However, owing to high energy and intensity, it is easy for the aforesaid light to cause injury to cells. Therefore, general light sources or light-emitting diodes (LEDs) have been recently developed to replace the high-intensity light above. Due to the relatively low energy of light emitted from LEDs, appropriate illuminance of the light needs to be found to achieve the aforesaid effects. Too low illuminance of light does not induce good treatment and, conversely, too high illuminance of light injures cells and has to be generated by large LED devices. Accordingly, it is difficult to create a compact and portable LED device for phototherapy. In addition, the current light source is a unicolor illuminant.

Therefore, it is desirable to provide a photo-stimulation method and device with a light mixture, in which LEDs have different colors and are adjusted in a specific range of illuminance to promote collagen synthesis so that labor, power, and time cost can be economized and the skin condition of the treated patients can be improved.

### SUMMARY OF THE INVENTION

The object of the present invention is to provide a photo-stimulation method with a light mixture, in which LEDs are set to emit a combination of red and yellow light in a specific range of illuminance so as to stimulate collagen synthesis of fibroblasts and to promote blood circulation as well as speedup removal of dead cells.

To achieve the object, one aspect of the present invention provides a photo-stimulation method with a light mixture, including the following steps: providing a light-emitting diode (LED) illuminant which is a combination of a yellow LED and a red LED; and illuminating a subject by the LED illuminant to promote collagen synthesis, wherein the yellow LED is in an illuminance range from 1,000 to 3,500 lux, the red LED is in an illuminance range from 6,000 to 9,500 lux, and a ratio of the yellow LED to the red LED is 0.5-2:0.5-2.

In conventional methods, laser or intense pulsed light with different wavelengths are applied to treat acne and to stimulate fibroblasts of dermis for an increase in collagen synthesis. Because laser or intense pulsed light with high intensity has to be produced by large apparatuses, it is difficult for general consumers to have such large apparatus. Although there has been research for LEDs which are used as a light source for acne treatment and promotion of collagen synthesis, the influence of a light mixture and illuminance of LEDs on cells or bacteria is not studied in conventional research. Therefore, it is not confirmed that the conventional method can achieve the aforesaid effects with unspecified illuminance of light. On the contrary, in the method of the present invention, a yellow-red light mixture of LEDs used as illuminants is adjusted in corresponding ranges of illuminance to efficiently achieve stimulation of fibroblasts and promotion of collagen synthesis.

When sustained illumination is achieved with a light mixture of yellow and red LEDs at appropriate illuminance for a suitable period of time, macrophages are stimulated to secrete cytokines for induction of fibroblast division. Also, fibroblasts are stimulated to synthesize DNA and secrete fibroblast growth factors (FGFs) for collagen synthesis. If the subject is a cell in vivo, for example, a fibroblast in dermis or a macrophage, light illumination on the skin can directly induce wound healing and anti-aging effects. Alternatively, if the subject is a cell in vitro, the cell can be treated as mentioned above and then implanted into animals for the aforesaid benefits. Accordingly, the subject of the present invention refers to a photo-stimulated subject.

In the photo-stimulation method of the present invention, the subject is preferably a fibroblast, a macrophage, or a combination thereof. In a preferred example of the present invention, the subject is a fibroblast. Furthermore, the wavelength of the yellow LED can range from 570 to 590 nm and the wavelength of the red LED can range from 620 to 750 nm. The illuminating time of a light mixture of yellow and red LEDs is not limited as long as the aforesaid benefits occur in the subject and the light illumination is not harmful thereto. The illuminating time can be adjusted according to the predetermined illuminance of light emitted from the yellow and red LEDs. If the light illuminance is relatively high, the benefits can be achieved in a relatively short period of illuminating time. Conversely, if the light illuminance is relatively low, the benefits can be carried out over a relatively long period of illuminating time.

For example, a yellow-red light mixture in which red LEDs light is in an illuminance range from 6,000 to 9,500 lux and yellow LEDs light is in an illuminance range from 1,000 to 3,500 lux, can illuminate the subject for 5-90 minutes. When a light mixture beyond the aforementioned range of illuminance, is used for illumination, the subject is not influenced by light illumination for a short period of time but is injured by light illumination for a long period of time due to an overdose of illuminance. Conversely, if a light mixture at an illuminance lower than the range of illuminance is used for illumination, the benefits are not achieved even under light illumination for a long period of time.

Another object of the present invention is to provide a photo-stimulation device with a light mixture. In the device, LEDs are set to emit a red-yellow light mixture in a specific range of illuminance so as to stimulate collagen synthesis of fibroblasts and to promote blood circulation as well as speed up removal of dead cells.

In order to achieve the object, another aspect of the present invention provides a photo-stimulation device with a light mixture. The device includes: a casing forming a deposition space and having a top surface and a lateral surface, wherein the top surface is provided with a light-output window; a diffuser plate covering the light-output window of the casing; a first illuminant module located in the deposition space of the casing and having a first light-emitting diode (LED) located under the diffuser plate, and the first light-emitting diode being a combination of red and yellow LEDs, wherein the light emitted from the yellow LED and passing through the diffuser plate has an illuminance in a range of 1,000-3,500 lux, the light emitted from the red LED and passing through the diffuser plate has an illuminance in a range of 6,000-9,500 lux, and a ratio of the yellow LED to the red LED is 0.5-2:0.5-2; and a controller module electrically connected with the first illuminant module and a power module.

In the photo-stimulation device of the present invention, a combination of yellow and red LEDs is used as an illuminant with a light mixture in corresponding ranges of illuminance. Once a subject is illuminated by the photo-stimulation device of the present invention, fibroblasts can be stimulated to synthesize collagen.

In the photo-stimulation device of the present invention, the power module can be an external power supply or be placed in the deposition space of the casing. The power module can contain rechargeable or dry batteries or microbatteries placed in the deposition space of the casing. Alternatively, if the power module is an external power supply or a rechargeable battery placed in the deposition space of the casing, the controller module can selectively further include a charge socket that provides an electrical connection between the power module and the controller module.

In the photo-stimulation device of the present invention, the controller module can selectively comprise a power switch mounted on the surface of the casing to control power output of the power module. Furthermore, the casing is preferably made of a material with low transmittance, for example, a material with high reflectivity or density such that light leakage of the photo-stimulation device can be prevented. Also, in order to prevent light leakage of the photo-stimulation device, one skilled in the art of the present invention can increase tightness of the whole device by various structural designs.

In the photo-stimulation device of the present invention, the lateral surface of the casing can be selectively provided with a light-output hole. In this case, the photo-stimulation device can further include a light-transmission plate covering the light-output hole, and a second illuminant module deposed corresponding to the light-transmission plate and emitting light which passes through the light-transmission plate. In this case, the controller module can further include a mode switch mounted on the surface of the casing to turn on the first illuminant module or the second illuminant module, i.e. to switch between the first illuminant module and the second illuminant module. In the first and second illuminant modules, assigned LEDs can be in the same color or different colors.

In the photo-stimulation device of the present invention, the diffuser plate placed on the light-output window is beneficial for uniform light emission and to avoid direct light illumination on users' eyes. Also, uniformity of photo-stimulation of the device can be increased. In other words, light of the LEDs is classified into a point source passing through the diffuser plate and then forming a surface light at the light-output window. The light-transmission plate placed on the light-output hole does not have to be a diffuser plate. If the light-transmission plate is a diffuser plate, the benefits described above can be achieved. If the light-transmission plate is not a diffuser plate, light supplied by a point light source can be directly transmitted.

In the photo-stimulation device of the present invention, the first and second illuminant modules, which red and yellow LEDs constitute, can be designed as being replaceable. If there is a need of red-light illumination in a relatively high ratio, the illuminant module in which the number of red LEDs is increased can be used. Furthermore, LEDs used in the first and second illuminant modules can be designed as being replaceable. In other words, if there is a need of yellow-light illumination in a relatively high ratio, the number of yellow LEDs used in the illuminant module can be increased.

In conclusion, the photo-stimulation method and device with a light mixture can employ LEDs with different colors such as yellow and red LEDs for photo-stimulation. Therefore, promotion of collagen synthesis can be achieved so as to carry out skin whitening or anti-aging benefits.

### BRIEF DESCRIPTION OF THE DRAWINGS

FIG. 1 is a perspective view of a photo-stimulation device in Example 1 of the present invention;

FIG. 2 is a side view of a photo-stimulation device in Example 1 of the present invention;

FIG. 3 is a system block diagram of a photo-stimulation device in Example 1 of the present invention;

FIG. 4 is a chart of human fibroblast viability and collagen synthesis in Example 2 of the present invention;

FIG. 5 is a chart of collagen synthesis percent of per unit human fibroblasts in Example 2 of the present invention;

FIG. 6 is a chart of human fibroblast viability and collagen synthesis in Example 3 of the present invention; and

FIG. 7 is a chart of collagen synthesis percent of per unit human fibroblasts in Example 3 of the present invention.

### DETAILED DESCRIPTION OF THE PREFERRED EMBODIMENT

Because of the specific embodiments illustrating the practice of the present invention, one skilled in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

The drawings of the embodiments in the present invention are all simplified charts or views, and only reveal elements relative to the present invention. The elements revealed in the drawings are not necessarily aspects of the practice, and quantity and shape thereof are optionally designed. Further, the design aspect of the elements can be more complex.

### Example 1

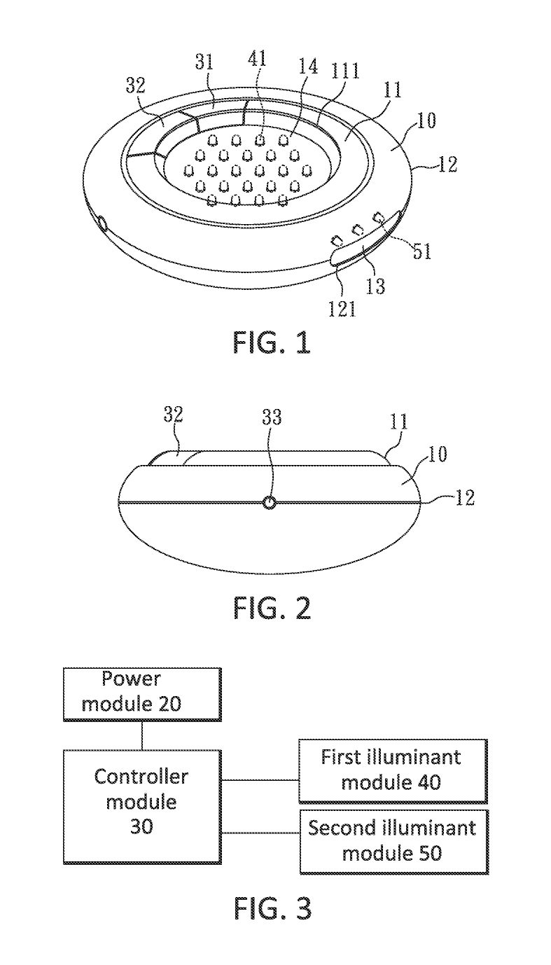

With reference to FIGS. 1 to 3, FIGS. 1 to 3, respectively, show a perspective view, a side view, and a system block diagram of a photo-stimulation device with a light mixture according to the present invention.

As shown in FIGS. 1 to 3, the photo-stimulation device of the present invention includes: a casing 10, a diffuser plate 14, a light-transmission plate 13, a first illuminant module 40, a second illuminant module 50, and a controller module 30.

The casing 10 forms a deposition space for receiving different modules. In addition, the casing 10 has a top surface 11 and a lateral surface 12. The top surface 11 is provided with a light-output window 111. The lateral surface 12 is provided with a light-output hole 121.

The light-output window 111 of the top surface 11 is covered by the diffuser plate 14, and the light-output hole 121 of the lateral surface 12 is covered by the light-transmission plate 13. The second illuminant module 50 corresponds to the light-transmission plate 13 and is placed in the deposition space of the casing 10. The second illuminant module 50 emits light passing through the light-transmission plate 13 and has one or more second LEDs 51. Herein, if the light-transmission plate 13 is used for light transmittance but not for light diffusion, the second illuminant module 50 serves as a point source of light.

The first illuminant module 40 is located in the deposition space of the casing 10 and a plurality of first LEDs 41 are arranged in an array under the diffuser plate 14. The first LEDs 41 are selected from a group consisting of a red LED, a yellow LED, and a blue LED. The light passing through the diffuser plate and emitted from the yellow and red LED has an illuminance in a range of 1,000-3,500 lux and 6,000-9,500 lux, respectively. In addition, the number of the yellow and red LEDs is in a ratio of 0.5-2:0.5-2. In the present example, the number of the yellow and red LEDs is at a ratio of 1:1.

The controller 30 is electrically connected with the first illuminant module 40 and a power module 20, and includes: a charge socket 33 which provides an electrical connection between the power module 20 and the controller module 30; a power switch 31 mounted on the surface of the casing 10 to control power output of the power module 20; and a mode switch 32 mounted on the surface of the casing 10 to turn on the first illuminant module 40 or the second illuminant module 50.

The power module 20 can be an external power supply or is placed in the deposition space of the casing 10. When the power module 20 is placed in the deposition space of the casing 10, the power module 20 can contain rechargeable or dry batteries or microbatteries for power supply.

Accordingly, in the photo-stimulation device with a light mixture, red and yellow LEDs that emit a light mixture in a specific range of illuminance are employed to stimulate fibroblasts and collagen synthesis and to promote blood circulation as well as speed up removal of dead cells.

### Example 2

LEDs that emitted red light at 7,800 lux were used to illuminate human fibroblasts. The influence of light illumination on the viability and collagen synthesis of the fibroblasts was studied.

First, human fibroblasts (2×10⁴ cells/well) were seeded with DMEM in a 48-well plate and cultured for 24 hours in a CO₂ incubator. Each well of the 48-well plate contained the cells and DMEM in a total volume of 0.5 ml. Subsequently, all the culture media were removed and then PBS (0.5 ml) was added to each well. The cells were illuminated by red LEDs (7,800 lux) for 5, 10, 15, and 30 minutes. Then, total PBS in the well was removed and DMEM (0.5 ml) was added to each well. The cells were incubated for another 24 hours and then photo-stimulated again according to the method mentioned above.

The culture medium in each well was replaced with flash DMEM (0.5 ml) and MTT reagent (0.125 ml) was added to each well. Then, the cells were incubated in an incubator (5% CO₂, 37° C.) for 4 hours. The culture media were totally collected and formazan (dissolved in DMSO, 0.5 ml) was added to the collected media. After reaction, the mixtures (0.2 ml) were analyzed in a 96-well plate by an ELISA Reader (SpectraMax M2) and absorbance thereof was measured at 570 nm. The cell viability was calculated according to the following equation where the control referred to cells that were not illuminated by the photo-stimulation device.

Cell viability(%)=(illuminated OD₅₇₀/control OD₅₇₀)×100%

In addition, collagen analysis was performed as follows. First, human fibroblasts (2×10⁴ cells/well) were seeded with DMEM in a 48-well plate and cultured for 24 hours in a CO₂ incubator. Each well of the 48-well plate contained the cells and DMEM in a total volume of 0.5 ml. Subsequently, all the culture media were removed and then PBS (0.5 ml) was added to each well. The cells were illuminated for 5, 10, 15, and 30 minutes by red LEDs (7,800 lux). Then, total PBS in the well was removed and DMEM (0.5 ml) was added to each well. The cells were incubated for another 24 hours and then photo-stimulated again according to the method mentioned above.

Subsequently, the culture media were totally collected in Eppendorf tubes (1.5 ml). An aqueous solution of acetic acid (0.5 M, 0.5 ml, 4° C.) was added to each well and stood for 20 minutes to dissolve the collagen. The solution of each well was collected in an Eppendorf tube. Then, acid neutralizing reagent (50 μl, Biocolor) and isolation & concentration reagent (4° C., 100 μl, Biocolor) were added to the Eppendorf tubes in sequence. The mixture stood at 4° C. overnight, and was then centrifugated at 12,000 rpm for 10 minutes. The supernatant was removed. Then, sircol dye reagent (1 ml, Biocolor) was added to the tubes. The tubes were sonicated for 30 minutes and centrifugated at 12,000 rpm for 10 minutes. The supernatant was removed. Subsequently, acid-salt wash reagent (4° C., 750 μl, Biocolor) was added to the tubes. The tubes were centrifugated at 12,000 rpm for 10 minutes. The supernatant was removed. Then, alkali reagent (250 μl, Biocolor) was added to the tubes. The mixture (200 μl) of each tube was taken out and added to each well of a 96-well plate. The absorbance of the mixtures was measured at 570 nm.

Collagen synthesis rate(%)=(Collagen synthesis after illumination/Collagen synthesis of control)×100%

In the equation, the control referred to cells that were not illuminated by the photo-stimulation device.

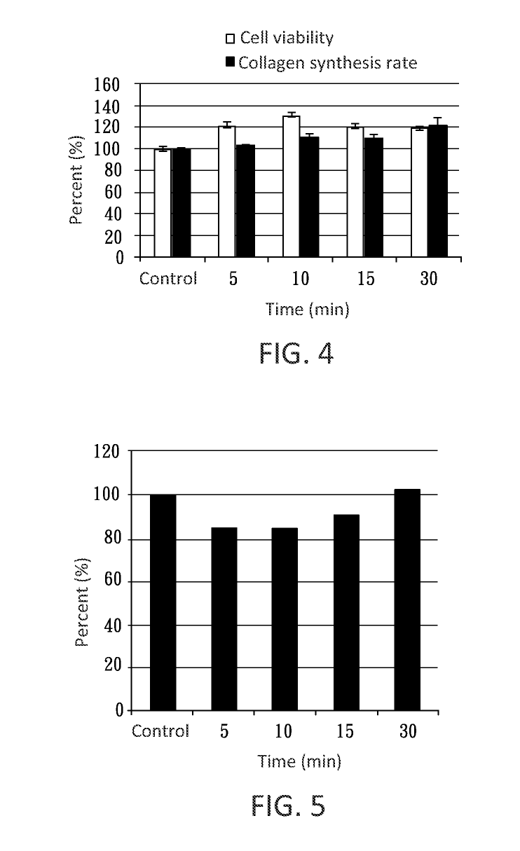

The results are shown in FIGS. 4 and 5. FIG. 4 is a chart of viability and collagen synthesis of human fibroblast illuminated by red LEDs at 7,800 lux. FIG. 5 is a chart of collagen synthesis percent of per unit the illuminated human fibroblasts. As shown in FIG. 4, after illumination for 5 minutes, fibroblast proliferation is promoted. The amount of collagen is also increased with an increase in fibroblasts. In addition, as shown in FIG. 5, although the amount of collagen synthesis percent of per unit the fibroblasts lowers (as compared with the control) owing to an increase in the number of fibroblasts in the beginning, the amount of collagen synthesis percent of per unit the fibroblasts gradually increases with the prolongation of illuminating time.

### Example 3

LEDs that emitted yellow light at 2,290 lux were used to illuminate human fibroblasts. The influence of light illumination on the viability and collagen synthesis of the fibroblasts was studied.

First, human fibroblasts (2×10⁴ cells/well) were seeded with DMEM in a 48-well plate and cultured for 24 hours in a CO₂ incubator. Each well of the 48-well plate contained the cells and DMEM in a total volume of 0.5 ml. Subsequently, all the culture media were removed and then PBS (0.5 ml) was added to each well. The cells were illuminated by yellow LEDs (2,290 lux) for 5, 10, 15, and 30 minutes. Then, total PBS in the well was removed and DMEM (0.5 ml) was added to each well. The cells were incubated for another 24 hours and then photo-stimulated again according to the method mentioned above.

The culture medium in each well was replaced with flash DMEM (0.5 ml) and MTT reagent (0.125 ml) was added to each well. Then, the cells were incubated in an incubator (5% CO₂, 37° C.) for 4 hours. The culture media were totally collected and formazan (dissolved in DMSO, 0.5 ml) was added to the collected media. After reaction, the mixtures (0.2 ml) were analyzed in a 96-well plate by an ELISA Reader (SpectraMax M2) and absorbance thereof was measured at 570 nm. The cell viability was calculated according to the following equation where the control referred to cells that were not illuminated by the photo-stimulation device.

Cell viability(%)=(illuminated OD₅₇₀/control OD₅₇₀)×100%

In addition, collagen analysis was performed as follows. First, human fibroblasts (2×10⁴ cells/well) were seeded with DMEM in a 48-well plate and cultured for 24 hours in a CO₂ incubator. Each well of the 48-well plate contained the cells and DMEM in a total volume of 0.5 ml. Subsequently, all the culture media were removed and then PBS (0.5 ml) was added to each well. The cells were illuminated for 5, 10, 15, and 30 minutes by yellow LEDs (2,290 lux). Then, total PBS in the well was removed and DMEM (0.5 ml) was added to each well. The cells were incubated for another 24 hours and then photo-stimulated again according to the method mentioned above.

Subsequently, the culture media were totally collected in Eppendorf tubes (1.5 ml). An aqueous solution of acetic acid (0.5 M, 0.5 ml, 4° C.) was added to each well and stood for 20 minutes to dissolve the collagen. The solution of each well was collected in an Eppendorf tube. Then, acid neutralizing reagent (50 μl, Biocolor) and isolation & concentration reagent (4° C., 100 μl, Biocolor) were added to the Eppendorf tubes in sequence. The mixture stood at 4° C. overnight, and was then centrifugated at 12,000 rpm for 10 minutes. The supernatant was removed. Then, sircol dye reagent (1 ml, Biocolor) was added to the tubes. The tubes were sonicated for 30 minutes and centrifugated at 12,000 rpm for 10 minutes. The supernatant was removed. Subsequently, acid-salt wash reagent (4° C., 750 μl, Biocolor) was added to the tubes. The tubes were centrifugated at 12,000 rpm for 10 minutes. The supernatant was removed. Then, alkali reagent (250 μl, Biocolor) was added to the tubes. The mixture (200 μl) of each tube was taken out and added to each well of a 96-well plate. The absorbance of the mixtures was measured at 570 nm.

Collagen synthesis rate(%)=(Collagen synthesis after illumination/Collagen synthesis of control)×100%

In the equation, the control referred to cells that were not illuminated by the photo-stimulation device.

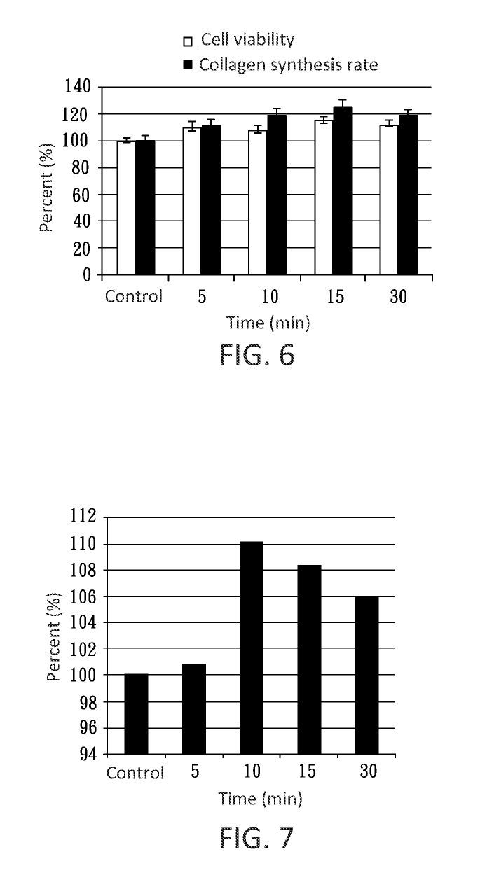

The results are shown in FIGS. 6 and 7. FIG. 6 is a chart of viability and collagen synthesis percent of per unit human fibroblast illuminated by yellow LEDs at 2,290 lux. FIG. 7 is a chart of collagen synthesis percent of per unit the illuminated human fibroblasts. As shown in FIG. 6, after illumination for 5 minutes, fibroblast proliferation is promoted. The amount of collagen is also increased with an increase in fibroblasts. In addition, as shown in FIG. 7, after illumination for about 5-10 minutes, the amount of collagen synthesis percent of per unit the fibroblasts is increased to the maximum.

Although the present invention has been explained in relation to its preferred embodiment, it is to be understood that many other possible modifications and variations can be made without departing from the spirit and scope of the invention as hereinafter claimed.

1-4. (canceled)

5. A photo-stimulation device with a light mixture, comprising:
a casing forming a deposition space and having a top surface and a lateral surface, wherein the top surface is provided with a light-output window;
a diffuser plate covering the light-output window of the casing;
a first illuminant module located in the deposition space of the casing and having a first light-emitting diode (LED) located under the diffuser plate, and the first LED being a combination of a yellow LED and a red LED, wherein the light emitted from the yellow LED and passing through the diffuser plate has an illuminance in a range of 1,000-3,500 lux, the light emitted from the red LED and passing through the diffuser plate has an illuminance in a range of 6,000-9,500 lux, and a ratio of the yellow LED to the red LED is 0.5-2:0.5-2;
a controller module electrically connected with the first illuminant module and a power module;
a light-transmission plate covering the light-output hole; and
a second illuminant module deposed corresponding to the light-transmission plate and emitting light which passes through the light-transmission plate;
wherein the lateral surface of the casing is provided with a light-output hole.

6. The photo-stimulation device of claim 5, wherein the power module is an external power supply or is placed in the deposition space of the casing.

7. The photo-stimulation device of claim 6, wherein the controller module comprises a charge socket which provides an electrical connection between the power module and the controller module.

8. The photo-stimulation device of claim 5, wherein the controller module comprises a power switch mounted on the surface of the casing to control power output of the power module.

9-10. (canceled)

11. The photo-stimulation device of claim 5, wherein the controller module comprises a mode switch mounted on the surface of the casing to turn on the first illuminant module or the second illuminant module.
